(19) United States
(12) Patent Application Publication
(10) Pub. No.: US 2022/0153739 A1
(43) Pub. Date: May 19, 2022

(54) OPIOID RECEPTOR MODULATOR AND USE THEREOF

(71) Applicant: Arriba BioPharma, Inc., Palo Alto, CA (US)

(72) Inventors: Xiaoqi CHEN, Palo Alto, CA (US); Hing Leung Sham, Palo Alto, CA (US)

(21) Appl. No.: 17/591,538

(22) Filed: Feb. 2, 2022

Related U.S. Application Data
(63) Continuation of application No. PCT/US2021/038546, filed on Jun. 22, 2021.
(60) Provisional application No. 63/042,636, filed on Jun. 23, 2020.

(51) Int. Cl. C07D 471/08; C07D 221/28

(52) U.S. Cl. CPC C07D 471/08; C07D 221/28

(57) ABSTRACT

The present disclosure relates to novel opioid receptor modulator compounds. Pharmaceutical compositions comprising such buprenorphine analog compound are also disclosed, and the use of such compound in the treatment of visceral hyperalgesia, diarrhea-predominant irritable bowel syndrome, and short bowel syndrome. The opioid receptor modulator compounds of the disclosure include Compound D.

### CROSS-REFERENCE TO RELATED APPLICATIONS

This application is a bypass continuation of PCT/US2021/038546, filed on Jun. 22, 2021, which claims priority to U.S. provisional application No. 63/042,636, filed on Jun. 23, 2020, the disclosures of which are incorporated by reference in its entirety herein.

### BACKGROUND

Opioid receptors are a group of inhibitory G protein-coupled receptors with opioids as ligands. The endogenous opioids are dynorphins, enkephalins, endorphins, endomorphins and nociceptin. The opioid receptors are ˜40% identical to somatostatin receptors (SSTRs). Opioid receptors are distributed widely in the brain, in the spinal cord, on peripheral neurons, and digestive tract. To date, five types of opioid receptors have been discovered-mu receptor (MOR), kappa receptor (KOR), delta receptor (DOR), nociception receptor (NOR) and zeta receptor (ZOR). Within these different types are a subset of subtypes, mu1, mu2, mu3, kappa1, kappa2, kappa3, delta1, and delta2.

### SUMMARY

Provided herein is a compound that is useful for binding and modulating μ, κ, and/or δ opioid receptors. The disclosure also provides composition, including pharmaceutical compositions, kits that include the compound, and methods of using (or administering) and making the compound. The disclosure further provides compound or compositions thereof for use in a method of treating a disease, disorder, or condition that is mediated by μ, κ, and δ opioid receptors. Moreover, the disclosure provides uses of the compound or compositions thereof in the manufacture of a medicament for the treating of a disease, disorder or condition that is mediated, at least in part, by μ, κ, and δ receptors.

One aspect of the present disclosure provides a compound of Formula I, or a pharmaceutically acceptable salt, hydrate, solvate, or prodrug thereof.

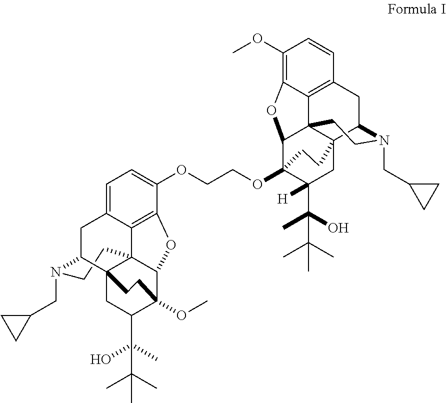

In some embodiments, the compound is in the form of a pharmaceutically acceptable salt.

In some embodiments, the compound is in the form of a hydrochloric acid salt.

In some embodiments, the compound is an opioid receptor modulator.

In some embodiments, the compound is a partial agonist of a μ opioid receptor.

In some embodiments, the compound is an antagonist of a κ opioid receptor.

In some embodiments, the compound is a partial agonist of a μ opioid receptor and an antagonist of a κ opioid receptor.

Another aspect of the present disclosure provides a pharmaceutical composition comprising a pharmaceutically acceptable carrier or excipient and the compound of Formula I, or a pharmaceutically acceptable salt, hydrate, solvate, or prodrug thereof.

In some embodiments, the composition is formulated for oral administration.

Another aspect of the present disclosure provides the use of the compound of Formula I, or a pharmaceutically acceptable salt, hydrate, solvate, or prodrug thereof in the treatment, reduction of severity, or alleviation of symptom of a disease or condition associated with at least one of μ, κ, and δ opioid receptors.

In some embodiments, the use of the compound of Formula I, or a pharmaceutically acceptable salt, hydrate, solvate, or prodrug thereof is for the treatment, reduction of severity, or alleviation of symptom of a disease or condition associated with at least one of μ and κ opioid receptors.

In some embodiments, the treatment, reduction of severity or alleviation of symptom of the disease or condition is by inhibiting a κ opioid receptor.

In some embodiments, the treatment, reduction of severity or alleviation of symptom of the disease or condition is by activating a μ opioid receptor.

In some embodiments, the treatment, reduction of severity or alleviation of symptom of the disease or condition is by inhibiting a κ opioid receptor and activating a μ opioid receptor.

Another aspect of the present disclosure provides the use of the compound of Formula I, or a pharmaceutically acceptable salt, hydrate, solvate, or prodrug thereof in the treatment, reduction of severity, or alleviation of symptom of a disease or condition selected from the group consisting of BS-D, visceral hyperalgesia, short bowel syndrome, or combinations thereof.

In some embodiments, the disease or condition is IBS-D.

In some embodiments, the disease or condition is visceral hyperalgesia.

In some embodiments, the disease or condition is short bowel syndrome.

In some embodiments, the compound is administered to a subject in need thereof at about 0.1 mg/kg of body weight to about 7.2 mg/kg of body weight.

In some embodiments, the compound is administered to a subject in need thereof at about 10 to about 500 mg daily.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates the results of μ opioid receptor binding assay (left) and κ opioid receptor binding assay (right) for Compound D;

FIG. 2 illustrates the results of μ opioid receptor FLIPR assay (left) and κ opioid receptor FLIPR assay (right) for Compound D;

FIG. 3 illustration results of in vivo testing (Induced Fecal Output) of Compound D, compared to vehicle; and

FIG. 4 illustrates in vivo testing (Post-Inflammatory Altered GI Transit Time) of Compound D, compared to vehicle.

### DETAILED DESCRIPTION

Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

In some embodiments, the compounds disclosed herein contain one or more asymmetric centers and thus give rise to enantiomers, diastereomers, and other stereoisomeric forms that are defined, in terms of absolute stereochemistry, as (R) or (S). Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). In some embodiments, acid addition salts of basic compounds are prepared by contacting the free base forms with a sufficient amount of the desired acid to produce the salt according to methods and techniques with which a skilled artisan is familiar.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

“Prodrug” is meant to indicate a compound that is converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7 9, 21 24 (Elsevier, Amsterdam).

A discussion of prodrugs is provided in Higuchi, T., et al., “Prodrugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers, which release the active compound in vivo when such prodrug is administered to a mammalian subject. In some embodiments, prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

Compounds

According to one aspect of the present disclosure, an opioid receptor modulator is provided to treat diseases or conditions associated with at least one of μ, κ, and δ opioid receptors. In some embodiments, the diseases or conditions include, but are not limited to, Irritable Bowel Syndrome (e.g. IBS-D). In some embodiments, the opioid receptor modulator disclosed herein is less CNS permeating that buprenorphine. In some embodiments, the opioid receptor modulator disclosed herein is non-CNS permeating.

In some embodiment, the opioid receptor modulator used in present disclosure has the following structure (Formula I), which is also referred to as “Compound D” or “ALB-215896”. In some embodiment, the opioid receptor modulator used in present disclosure is a pharmaceutically acceptable salt of Compound D. In some embodiment, the opioid receptor modulator used in present disclosure is a prodrug of Compound D. In some embodiment, the prodrug of Compound D is obtained by esterification of one or both of the free hydroxy groups in Compound D (e.g. acetylation). In some embodiment, the opioid receptor modulator used in present disclosure is a derivative of Compound D, such as by converting one or both of the methoxy group attached to the phenyl ring to hydroxy group(s).

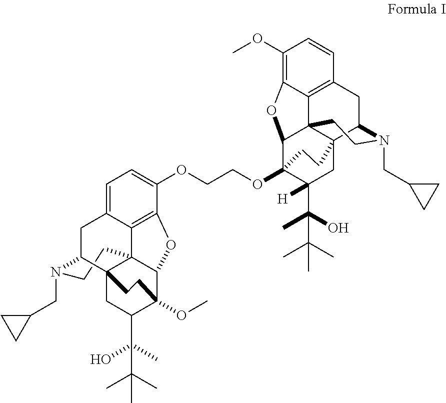

Pharmaceutical Compositions

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, Pa. (2005)), the disclosure of which is hereby incorporated herein by reference in its entirety.

Accordingly, provided herein is a pharmaceutical composition comprising at least one compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (A), or a pharmaceutically acceptable salt thereof.

One embodiment provides a pharmaceutical composition comprising a pharmaceutically acceptable carrier and a compound of Formula (B), or a pharmaceutically acceptable salt thereof.

One embodiment provides a pharmaceutical composition comprising a pharmaceutically acceptable carrier and a compound of Formula (C), or a pharmaceutically acceptable salt thereof.

Another embodiment provides a pharmaceutical composition consisting essentially of a pharmaceutically acceptable carrier and a compound of Formula (A), or a pharmaceutically acceptable salt thereof. Another embodiment provides a pharmaceutical composition consisting essentially of a pharmaceutically acceptable carrier and a compound of Formula (B), or a pharmaceutically acceptable salt thereof. Another embodiment provides a pharmaceutical composition consisting essentially of a pharmaceutically acceptable carrier and a compound of Formula (C), or a pharmaceutically acceptable salt thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid or liquid form, which includes one or more of a disclosed compound, as an active ingredient, in admixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component is optionally mixed under sterile conditions with a pharmaceutically acceptable carrier, and with any preservatives, buffers, or propellants which are required in some embodiments.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. Sprays additionally contain customary propellants, such as chlorofluorohydrocarbons and volatile unsubstituted hydrocarbons, such as butane and propane.

Compositions and compounds disclosed herein are alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are used because they minimize exposing the agent to shear, which result in degradation of the compounds contained in the subject compositions in some embodiments. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Examples of suitable aqueous and non-aqueous carriers employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. In some embodiments, proper fluidity is maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

In some embodiments, the dose of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

In some embodiments, oral doses typically range from about 1.0 mg to about 1000 mg, one to four times, or more, per day.

### Opioid Receptors

MOP Receptor (“MOR”)

The MOP receptor was the last of the classical opioid receptors to be cloned and is located throughout the central nervous system in areas involved in sensory and motor function including regions concerned with the integration and perception of these senses, for example cerebral cortex, amygdala (of the limbic system). High density of MOP receptors is found in the caudate putamen (of the basal ganglia). MOP receptors are located presynaptically on primary afferent neurons within the dorsal horn of the spinal cord where they inhibit glutamate release and hence transmission of nociceptive stimuli from C and Ab fibres. The periaqueductal grey (PAG) is an area of the midbrain involved in the central control of nociceptive transmission. Efferent outflow from the PAG descends to the spinal cord where it acts to inhibit nociceptive transmission in afferent fibres, this pathway is known as the descending inhibitory control pathway. High densities of MOP receptor are found in the PAG and the analgesia of some opioids is proposed to come about from removal of an inhibitory γ-amino butyric acid (GABA)-ergic tone in this region of the brain. GABA is the main inhibitory transmitter in the brain and acts to reduce or prevent antinociceptive outflow from the PAG.

Major side-effects associated with MOP agonists include respiratory depression through a reduction in the sensitivity of central and peripheral chemoreceptors to hypercapnia. MOP agonists further inhibit gastrointestinal tract secretions and peristalsis, often causing constipation. MOP opioids also have effects on the cardiovascular system, thermoregulation, hormone secretion and immune function.

Studies using MOP receptor knockout mice have defined the role MOP plays tonically and when stimulated by exogenously applied ligands. MOP receptor knockout mice show increased sensitivity to thermal pain, implicating the receptor in this mode of nociception. However, no change in threshold from pain elicited via mechanical stimuli was seen. None of the predicted effects or side-effects of morphine were seen in mice lacking the MOP receptor. There is no change in respiratory function demonstrating no tonic role in this system. Morphine did not produce analgesia or respiratory effects. This genetic approach confirms that both the wanted and unwanted effects of morphine are attributable to action at the MOP receptor. 4

Whilst the main analgesic effects of opioids are elicited by central activation of opioid receptors, a number of the common side-effects, including reduced gastrointestinal motility, urinary retention and pruritus, are regulated by activation of peripherally located opioid receptors. The use of peripherally acting opioid receptor antagonists may reduce a number of these peripherally mediated side-effects, for example methylnaltrexone, a peripherally acting opioid antagonist, was shown in clinical trials to be effective at treating opioid-induced constipation.

DOP Receptor (“DOR”)

The DOP receptor was the first to be cloned and is less widely distributed relative to the other opioid receptors. Highest densities are found in the olfactory bulb, cerebral cortex, nucleus accumbens and the caudate putamen. DOP receptors are located presynaptically on primary afferents where they inhibit the release of neurotransmitters. Through both spinal and supraspinal sites, the receptor is involved in the antinociceptive/analgesic actions of some opioids. However, DOP receptor agonists have also been shown to reduce gastrointestinal tract motility and cause respiratory depression. 5 Studies with DOP receptor knockout mice revealed that they display hyperlocomotor activity and it is assumed the receptor, under basal conditions, may dampen locomotor behaviour. DOP receptor knockout mice also displayed anxiogenic and depressive-like responses, suggesting that the receptor may act to regulate mood.

KOP Receptor (“KOR”)

The kappa or KOP receptor was the second of the opioid receptor family to be cloned. The prototypical agonist of the kappa receptor is the non-peptide benzomorphan ketocyclazocine, the actions of which have been shown to be distinct from those elicited by stimulation of the MOP receptor, for example sedation without marked effects on heart rate. Two synthetic KOP receptor agonists, spiradoline (U-62,066E) and enadoline (CI-977) have undergone clinical trials for their analgesic actions. 67 Whilst spiradoline produced promising analgesia in animals, clinical data shows that spiradoline produces adverse effects such as diuresis, sedation and dysphoria at doses lower than those needed for analgesic effects. Enadoline produced similar side-effects including sedation, confusion and dizziness along with increased urinary output and feelings of depersonalization. The side-effects elicited by these and other KOP receptor agonists have, to date, limited their effective clinical use. However, it has been shown recently that KOP agonists (e.g. enadoline) may have neuroprotective actions via their ability to inhibit post ischaemic glutamate release.

The advantage of the KOP receptor agonists over other opioid ligands is that they do not cause respiratory depression. It must also be mentioned that KOP agonists also display an anti-opioid action attenuating analgesia elicited by endogenously released or exogenously administered MOP agonists. It has been hypothesized that this action is caused by a distinct distribution of KOP receptors on primary cells located within the nucleus raphe magnus (NRM), a group of cell bodies situated in the midbrain. The output from the NRM forms part of the descending inhibitory control pathway acting to dampen nociceptive transmission at the level of the spinal cord. The NRM consists of primary and secondary cells whose axons terminate in the spinal cord. It is suggested that secondary cell firing facilitates nociceptive transmission, whilst primary cells inhibit it. Analgesia elicited by exogenously applied opioids is mainly via agonist activity at the opioid receptor MOP. It has been shown that MOP receptors are situated only at secondary cells of the NRM. Inhibition of these secondary cells via MOP receptor stimulation results in pre-synaptic inhibition of GABA-ergic input to primary cells leading to their disinhibition. This disinhibition, which equates to primary cell stimulation, results in analgesia elicited at the level of the spinal cord. KOP receptors are localised only on the primary cells of the NRM and the anti-analgesic effect of KOP receptor agonists is caused by inhibition of the primary cells thus preventing indirect stimulation mediated through the MOP receptor pathway.

In the NRM stimulation of primary cells is thought to induce analgesia via activation of descending inhibitory control pathways and release of endogenous opioids. MOP agonists cause analgesia via inhibiting secondary cells, the output from which is inhibitory (GABA) toward primary cells. Removal of this GABA-ergic tone disinhibits primary cells resulting in their activation and thus analgesia. KOP and NOP receptors are situated on primary cells and their anti-opioid action is from a direct inhibition of these cells, preventing MOP receptor mediated disinhibition. (Modified from Pan 20008).

In the NRM stimulation of primary cells is thought to induce analgesia via activation of descending inhibitory control pathways and release of endogenous opioids. MOP agonists cause analgesia via inhibiting secondary cells, the output from which is inhibitory (GABA) toward primary cells. Removal of this GABA-ergic tone disinhibits primary cells resulting in their activation and thus analgesia. KOP and NOP receptors are situated on primary cells and their anti-opioid action is from a direct inhibition of these cells, preventing MOP receptor mediated disinhibition. (Modified from Pan 20008).

NOP Receptor (“NOR”)

At the cellular level, N/OFQ produces similar actions to those of the classical opioids resulting in reduced neuronal excitability and inhibition of transmitter release. Initial studies concentrated on the role N/OFQ and NOP in pain. However, exogenous administration has been shown to have effects on locomotion, stress and anxiety, feeding, learning and memory, reward/addiction and urogenital activity.

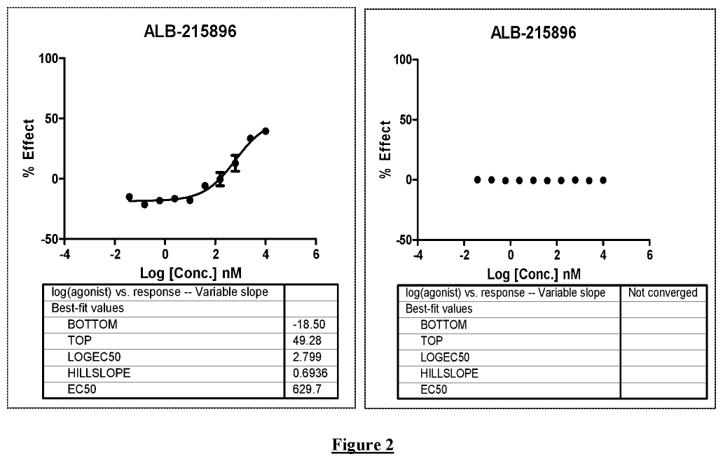

N/OFQ has been shown under laboratory conditions to have a pronociceptive, anti-analgesic effect when applied supraspinally whilst spinally N/OFQ causes analgesia at high doses; low doses lead to hyperalgesia. N/OFQ anti-analgesic action is the hypothesised cause for the supraspinal pronociception effect, inhibiting either endogenous opioid tone or stress-induced analgesia produced during testing procedures in laboratory animals. N/OFQ anti-opioid effect is caused by NOP receptor localization on, and inhibition of, primary cells of the NRM, analogous to the KOP receptor pathway (FIG. 2). 8 It is believed that endogenous levels of N/OFQ may act to set threshold to pain, as NOP receptor antagonists have been shown to give rise to a long lasting analgesia with similar efficacy to morphine. NOP receptor antagonists may have a possible future as novel analgesics or maybe used as an adjuvant to reduce the amount of classical opioid drug required to produce analgesia. Consequently, this may reduce the side-effects encountered when using classical opioids.

The N/OFQ-NOP system is believed to play a role in the development of tolerance to morphine analgesia. NOP receptor knockout mice show a partial loss of tolerance to morphine and there is an up regulation of N/OFQ production in chronic morphine tolerant mice. Studies in knockout mice confirmed that morphine tolerance to analgesia, but not acute response to morphine, was markedly attenuated. This action has also been confirmed through the actions of potent selective NOP antagonists, which also attenuate morphine tolerance. These findings suggest the N/OFQ-NOP system contributes to neuronal plasticity involved in the development of tolerance seen with chronic morphine exposure.

NOP receptors are localized on afferent fibres and N/OFQ has been evaluated for its acute urodynamic effects in patients suffering neurogenic detrusor overactivity incontinence. It was shown that N/OFQ elicited a robust acute inhibitory effect on the micturition reflex in this patient group. In summary, animal paradigms suggest that NOP receptor antagonists will be useful for the treatment of pain. Also, NOP receptor blockade may prove useful in reducing tolerance to opioids and/or reducing the dose of opioid ligand required to provide analgesia.

### Diseases and Conditions

In some embodiments, the opioid receptor modulator disclosed herein is used to treat a disease or condition associated with at least one of μ, κ, and δ opioid receptors. In some embodiments, the opioid receptor modulator disclosed herein is used to reduce severity of a disease or condition associated with at least one of μ, κ, and δ opioid receptors. In some embodiments, the opioid receptor modulator disclosed herein is used to alleviate symptoms of a disease or condition associated with at least one of μ, κ, and δ opioid receptors.

Irritable Bowel Syndrome

Irritable bowel syndrome (IBS) is characterized by recurrent abdominal pain and discomfort associated with alterations in the frequency or consistency of stool, that present as diarrhea or constipation. Based on the Rome III diagnostic criteria, which classify IBS according to different bowel behaviors, there are four subtypes: IBS-D (diarrhea-predominant), IBS-C (constipation-predominant), IBS-M (mixed), and unspecified IBS (IBS-U). Currently, there is no drug has been approved in the United States for chronic, unrestricted treatment IBS-D. Clinically, the complexity and diversity of IBS presentation make treatment difficult. In practice, clinicians generally make treatment decisions for symptom reduction in IBS according to the type and severity of the symptoms. Many pharmacological treatment approaches are associated with side effects that result in a decreased benefit to the patient in terms of treatment outcomes. Hence, IBS sufferers often have absenteeism, reduced health-related quality of life and multiple healthcare-seeking behaviors, leading to great social and economic burdens. Depending on the diagnostic criteria used to define the condition, it is reported to affect approximately 5%-20% of the general population worldwide.

In patients with IBS-D, there is increased colonic transit and enhanced peristaltic contractions, most notably after meals. The opioid receptors in the gut play a part in regulating gastrointestinal motility, secretions, and visceral sensations. There are several possible treatments for IBS-D, mainly focused on symptom management through lifestyle modification, psychotherapy, and pharmacotherapy. The treatment objective is to maintain everyday functioning and improve quality of life. Pharmacologic options for treatment of IBS-D are limited. There are several types of medications, including antispasmodic medicines, antidiarrheal and antidepressants.

In some embodiments, the opioid receptor modulator described here is useful in treating IBS-D. In some embodiments, the opioid receptor modulator described here is useful in reducing severity of IBS-D. In some embodiments, the opioid receptor modulator described here is useful in alleviating symptoms of IBS-D. In some embodiments, the opioid receptor modulator disclosed herein is less CNS permeating that buprenorphine. In some embodiments, the opioid receptor modulator disclosed herein is non-CNS permeating.

In some embodiments, the opioid receptor modulator used for treating IBS-D, reducing severity of IBS-D, or alleviating symptoms of IBS-D is a partial agonist of the μ receptors. In some embodiments, the opioid receptor modulator used for treating IBS-D, reducing severity of IBS-D, or alleviating symptoms of IBS-D is an agonist of the μ receptors. In some embodiments, the opioid receptor modulator used for treating IBS-D, reducing severity of IBS-D, or alleviating symptoms of IBS-D is an antagonist of the κ receptors. In some embodiments, the opioid receptor modulator used for treating IBS-D, reducing severity of IBS-D, or alleviating symptoms of IBS-D is a partial agonist of the μ receptors and an antagonist of the κ receptors.

Visceral Hyperalgesia

Visceral hyperalgesia is a higher sensitivity to the normal activity of organs inside the body. A person may notice normal intestinal activities that most people do not feel. These feelings may be painful. The pain when a person is sick is also stronger. It may be felt in the pancreas, intestines, and stomach. The type of pain can differ from person to person. It may be dull and achy, sharp, or burning pain. The pain may be all the time or it may come and go.

In many cases, visceral hyperalgesis, sometimes also referred to as visceral hypersensitivity, is associated with one or more of the following: infection of intestines and chronic GI diseases and disorders such as Irritable bowel syndrome (IBS), Crohn disease, Functional dyspepsia, and functional abdominal pain syndrome.

In some embodiments, the opioid receptor modulator described here is useful in treating visceral hyperalgesia. In some embodiments, the opioid receptor modulator described here is useful in reducing severity of visceral hyperalgesia. In some embodiments, the opioid receptor modulator described here is useful in alleviating symptoms of visceral hyperalgesia. In some embodiments, the opioid receptor modulator disclosed herein is less CNS permeating that buprenorphine. In some embodiments, the opioid receptor modulator disclosed herein is non-CNS permeating.

In some embodiments, the opioid receptor modulator used for treating visceral hyperalgesia, reducing severity of visceral hyperalgesia, or alleviating symptoms of visceral hyperalgesia is a partial agonist of the μ receptors. In some embodiments, the opioid receptor modulator used for treating visceral hyperalgesia, reducing severity of visceral hyperalgesia, or alleviating symptoms of visceral hyperalgesia is an agonist of the μ receptors. In some embodiments, the opioid receptor modulator used for treating visceral hyperalgesia, reducing severity of visceral hyperalgesia, or alleviating symptoms of visceral hyperalgesia is an antagonist of the κ receptors. In some embodiments, the opioid receptor modulator used for treating visceral hyperalgesia, reducing severity of visceral hyperalgesia, or alleviating symptoms of visceral hyperalgesia is a partial agonist of the μ receptors and an antagonist of the κ receptors.

Short Bowel Syndrome

Short bowel syndrome (SBS, or simply short gut) is a malabsorption disorder caused by a lack of functional small intestine. The primary symptom is diarrhea, which can result in dehydration, malnutrition, and weight loss. Other symptoms may include bloating, heartburn, feeling tired, lactose intolerance, and foul-smelling stool. Complications can include anemia and kidney stones.

Most cases are due to the surgical removal of a large portion of the small intestine. This is most often required due to Crohn's disease in adults and necrotising enterocolitis in young children. Other causes include damage to the small intestine from other means and being born with an abnormally short intestine. It usually does not develop until less than 2 in (6.6 ft) of the normally 6.1 n (20 ft) small intestine remains.

Treatment may include a specific diet, medications, or surgery. The diet may include slightly salty and slightly sweet liquids, vitamin and mineral supplements, small frequent meals, and the avoidance of high fat food. Occasionally nutrients need to be given through an intravenous line, known as parenteral nutrition. Medications used may include antibiotics, antacids, loperamide, teduglutide, and growth hormone. Different types of surgery, including an intestinal transplant, may help some people.

Short bowel syndrome newly occurs in about three per million people each year. There are estimated to be about 15,000 people with the condition in the United States. It is classified as a rare disease by the European Medicines Agency. Outcomes depend on the amount of bowel remaining and whether or not the small bowel remains connected with the large bowel.

In some embodiments, the opioid receptor modulator described here is useful in treating short bowel syndrome. In some embodiments, the opioid receptor modulator described here is useful in reducing severity of short bowel syndrome. In some embodiments, the opioid receptor modulator described here is useful in alleviating symptoms of short bowel syndrome. In some embodiments, the opioid receptor modulator disclosed herein is less CNS permeating that buprenorphine. In some embodiments, the opioid receptor modulator disclosed herein is non-CNS permeating.

In some embodiments, the opioid receptor modulator used for treating short bowel syndrome, reducing severity of short bowel syndrome, or alleviating symptoms of short bowel syndrome is a partial agonist of the μ receptors. In some embodiments, the opioid receptor modulator used for treating short bowel syndrome, reducing severity of short bowel syndrome, or alleviating symptoms of short bowel syndrome is an agonist of the μ receptors. In some embodiments, the opioid receptor modulator used for treating short bowel syndrome, reducing severity of short bowel syndrome, or alleviating symptoms of short bowel syndrome is an antagonist of the κ receptors. In some embodiments, the opioid receptor modulator used for treating short bowel syndrome, reducing severity of short bowel syndrome, or alleviating symptoms of short bowel syndrome is a partial agonist of the μ receptors and an antagonist of the κ receptors.

### NON-LIMITING EMBODIMENTS

Embodiment 1: A compound of Formula (I):

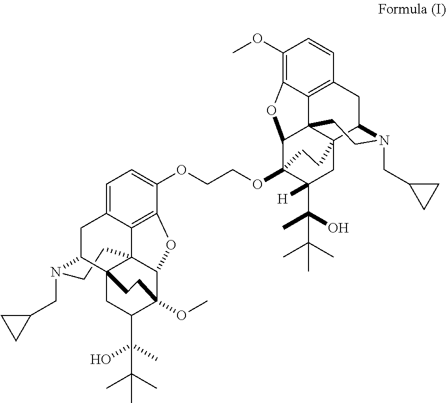

or a pharmaceutically acceptable salt, hydrate, solvate, or prodrug thereof.
Embodiment 2: The compound of Embodiment 1, wherein the compound is in the form of a pharmaceutically acceptable salt.
Embodiment 3: The compound of Embodiment 1, wherein the compound is in the form of a hydrochloric acid salt.
Embodiment 4: The compound of any one of Embodiments 1-3, wherein the compound is an opioid receptor modulator.
Embodiment 5: The compound of Embodiment 4, wherein the compound is a partial agonist of a μ opioid receptor.
Embodiment 6: The compound of Embodiment 4, wherein the compound is an antagonist of a κ opioid receptor.
Embodiment 7: The compound of Embodiment 4, wherein the compound is a partial agonist of a μ opioid receptor and an antagonist of a κ opioid receptor.
Embodiment 8: A pharmaceutical composition comprising a pharmaceutically acceptable carrier or excipient and the compound of any one of Embodiments 1-7.
Embodiment 9: The pharmaceutical composition of Embodiment 8, wherein said composition is formulated for oral administration.
Embodiment 10: The compound of any one of Embodiments 1-7 or the pharmaceutical composition of any one of Embodiments 8-9, for use in the treatment, reduction of severity, or alleviation of symptom of a disease or condition associated with at least one of μ, κ, and δ opioid receptors.
Embodiment 11: The compound or composition of Embodiment 10, for use in the treatment, reduction of severity, or alleviation of symptom of a disease or condition associated with at least one of μ and κ opioid receptors.
Embodiment 12: The compound or composition of Embodiment 11, wherein the treatment, reduction of severity or alleviation of symptom of the disease or condition is by inhibiting a κ opioid receptor.
Embodiment 13: The compound or composition of Embodiment 11, wherein the treatment, reduction of severity or alleviation of symptom of the disease or condition is by activating a μ opioid receptor.
Embodiment 14: The compound or composition of Embodiment 11, wherein the treatment, reduction of severity or alleviation of symptom of the disease or condition is by inhibiting a κ opioid receptor and activating a μ opioid receptor.
Embodiment 15: The compound or composition of any one of Embodiments 10-14, wherein the disease or condition is selected from the group consisting of IBS-D, visceral hyperalgesia, short bowel syndrome, or combinations thereof.
Embodiment 16: The compound or composition of any one of Embodiments 10-14, wherein the disease or condition is IBS-D.
Embodiment 17: The compound or composition of any one of Embodiments 10-14, wherein the disease or condition is visceral hyperalgesia.
Embodiment 18: The compound or composition of any one of Embodiments 10-14, wherein the disease or condition is short bowel syndrome.
Embodiment 19: The compound or composition of any one of Embodiments 10-18, characterized in that the compound is administered to a subject in need thereof at about 0.1 mg/kg of body weight to about 7.2 mg/kg of body weight.
Embodiment 20: The compound or composition of any one of Embodiments 10-19, characterized in that the compound is administered to a subject in need thereof at about 10 to about 500 mg daily.

### Examples

The disclosure is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1. Chemical Synthesis

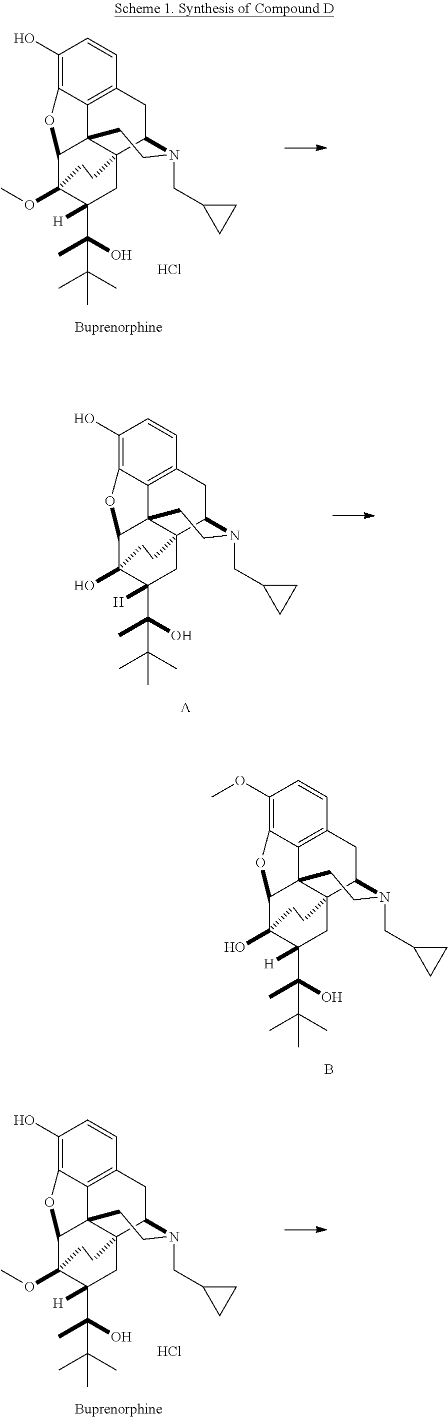

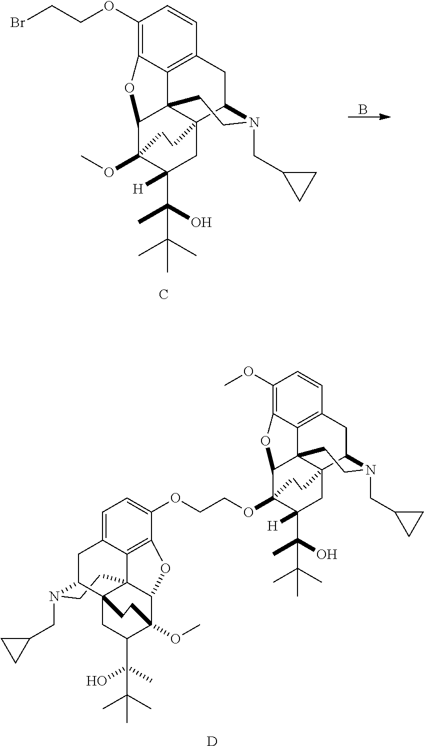

### 1, Synthesis of (4R,4aS,6R,7R,7aR,12bS)-3-(Cyclopropylmethyl)-6-(2-hydroxy-3,3dimethylbutan-2-yl)-1,2,3,4,5,6-hexahydro-4a,7-ethano-4,12-methanobenzofuro[3,2e]isoquinoline-7,9(7aH)-diol (A)

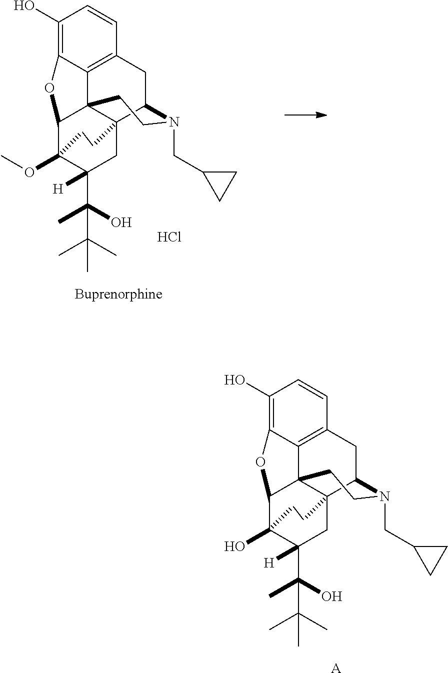

To a mixture of buprenorphine hydrochloride (550 mg, 1.09 mmol) in THE (10 mL) and CHCl₃ (10 mL) at 0° C. was added a solution of LAH (1 M THF, 4.4 mL, 4.4 mmol). After the addition, the reaction mixture was warmed to room temperature and heated at reflux for 12 h. The reaction mixture was cooled in an ice bath and quenched by water (0.8 mL), 15% of NaOH (0.8 mL) and water (2 mL). The mixture was stirred at room temperature for 15 min, and then magnesium sulfate powder was added. The solids were removed by filtration and rinsed with DCM. The filtrate was concentrated to dryness and purified by column chromatography (24 g, silica gel, 0 to 6% MeOH/DCM, gradient) to afford the title compound (490 mg, 99%) as a white solid. ¹H NMR (500 MHz, CDCl₃): δ 6.69 (d, J=8.0 Hz, 1H), 6.51 (d, J=8.0 Hz, 1H), 4.24 (s, 1H), 2.99 (s, 1H), 2.98 (d, J=9.5 Hz, 1H), 2.88-2.84 (m, 1H), 2.63-2.59 (m, 1H), 2.37-2.14 (m, 5H), 2.01-1.89 (m, 2H), 1.71-1.69 (m, 1H), 1.57 (s, 3H), 1.45 (s, 3H), 1.43-1.38 (m, 1H), 1.32-1.27 (m, 1H), 1.03 (s, 9H), 1.01-0.95 (m, 1H), 0.82-0.75 (m, 1H), 0.67-0.58 (m, 1H), 0.51-0.44 (m, 2H), 0.13-0.09 (m, 2H).

### 2. Synthesis of 2-((4R,4aS,6R,7R,7aR,12bS)-3-(Cyclopropylmethyl)-6-(2-hydroxy 3,3dimethylbutan-2-yl)-9-methoxy-1,2,3,4,5,6-hexahydro-4a,7-ethano-4,12methanobenzofuro[3,2-e]isoquinolin-7(7aH)-ol (B)

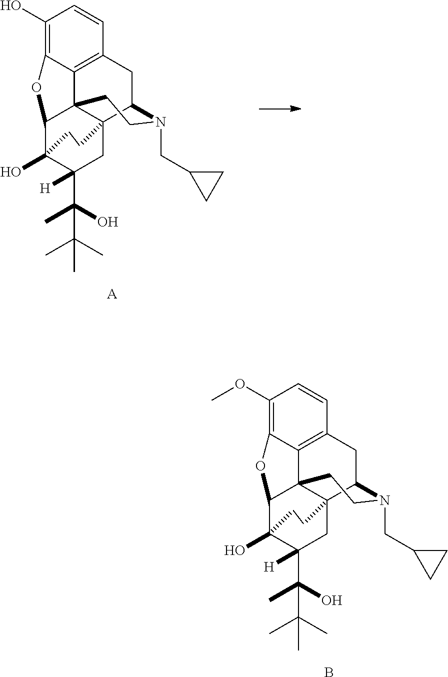

To a mixture of A (200 mg, 0.441 mmol), potassium carbonate (183 mg, 1.32 mmol) in DMSO (3 mL) was added iodomethane (0.036 mL, 0.573 mmol) at room temperature. After the addition, the mixture was stirred at room temperature for 12 h. The reaction mixture was diluted with EtOAc (100 mL). The organic phase was washed with brine, dried over magnesium sulfate, filtered, and concentrated to dryness. The crude material was purified by column chromatography (12 g, silica gel, 0 to 30% EtOAc/heptane, gradient) to afford the title compound (173 mg, 84%). ¹H NMR (500 MHz, CDCl₃): δ 6.71 (d, J=8.0 Hz, 1H), 6.58 (d, J=8.0 Hz, 1H), 4.86 (s, 1H), 4.25 (s, 1H), 3.87 (s, 3H), 3.06 (s, 1H), 3.02 (dd, J=11.5, 5.0 Hz, 2H), 2.87-2.82 (m, 1H), 2.63 (dd, J=12.0, 5.0 Hz, 1H), 2.37-2.15 (m, 5H), 2.04-1.95 (m, 2H), 1.71 (dd, J=12.5, 2.0 Hz, 1H), 1.40 (s, 3H), 1.39-1.30 (m, 2H), 1.24 (s, 9H), 1.03-0.95 (m, 1H), 0.83-0.78 (m, 1H), 0.68-0.58 (m, 1H), 0.51-0.47 (m, 2H), 0.12-0.10 (m, 2H).

### 3. Synthesis of 2-((4R,4aS,6R,7R,7aR,12bS)-9-(2-Bromoethoxy)-3-(cyclopropylmethyl)-7methoxy-1,2,3,4,5,6,7,7a-octahydro-4a,7-ethano-4,12-methanobenzofuro[3,2e]isoquinolin-6-yl)-3,3-dimethylbutan-2-ol (C)

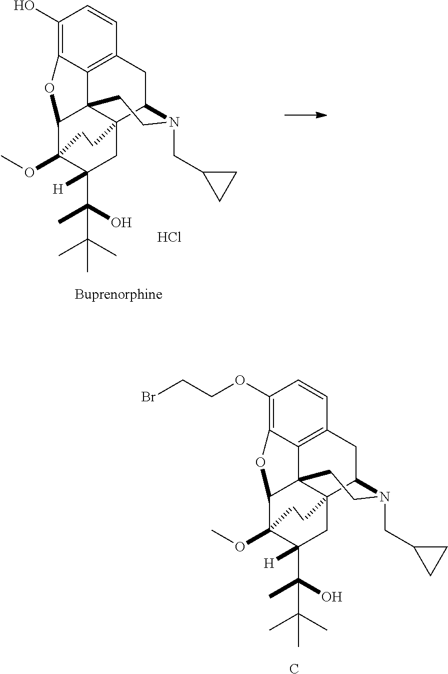

To a mixture of buprenorphine hydrochloride (400 mg, 0.79 mmol) and potassium carbonate (439 mg, 3.17 mmol) in DMSO (4 mL) at 60° C. was slowly added 1,2-dibromoethane (0.275 mL, 3.17 mmol). After the addition, the mixture was stirred at 60° C. for 16 h. The reaction mixture was cooled to room temperature and diluted with EtOAc (100 mL). The organic phase was washed with brine, dried over magnesium sulfate, filtered, and concentrated to dryness. The crude material was purified by column chromatography (silica gel, 0 to 30% EtOAc/heptane, gradient) to afford the title compound C (190 mg, 42%) and recovered buprenorphine (170 mg). ¹H NMR (500 MHz, CDCl₃): δ 6.75 (d, J=8.0 Hz, 1H), 6.54 (d, J=8.0 Hz, 1H), 5.88 (s, 1H), 4.45-4.31 (m, 3H), 3.78 (t, J=6.0 Hz, 2H), 3.54 (s, 3H), 2.99-2.97 (m, 2H), 2.90-2.88 (m, 1H), 2.62 (dd, J=11.5, 5.0 Hz, 1H), 2.36-2.14 (m, 5H), 1.97-1.95 (m, 1H), 1.84-1.76 (m, 2H), 1.68 (dd, J=13.0, 2.5 Hz, 1H), 1.35 (s, 3H), 1.43-1.41 (m, 2H), 1.03 (s, 9H), 0.83-0.66 (m, 2H), 0.51-0.47 (m, 2H), 0.12-0.11 (m, 2H).

### 4. Synthesis of 2-((4R,4aS,6R,7R,7aR,12bS)-9-(2-(((4R,4aS,6R,7R,7aS,12bS)-9-(Methyloxy)-3-(cyclopropylmethyl)-6-(2-hydroxy-3,3-dimethylbutan-2-yl)-1,2,3,4,5,6 hexahydro-4a,7-ethano-4,12-methanobenzofuro[3,2-e]isoquinolin-7(7aH)yl)oxy)ethoxy)-3-(cyclopropylmethyl)-7-methoxy-1,2,3,4,5,6,7,7a-octahydro-4a,7ethano-4,12-methanobenzofuro[3,2-e]isoquinolin-6-yl)-3,3-dimethylbutan-2-ol (D)

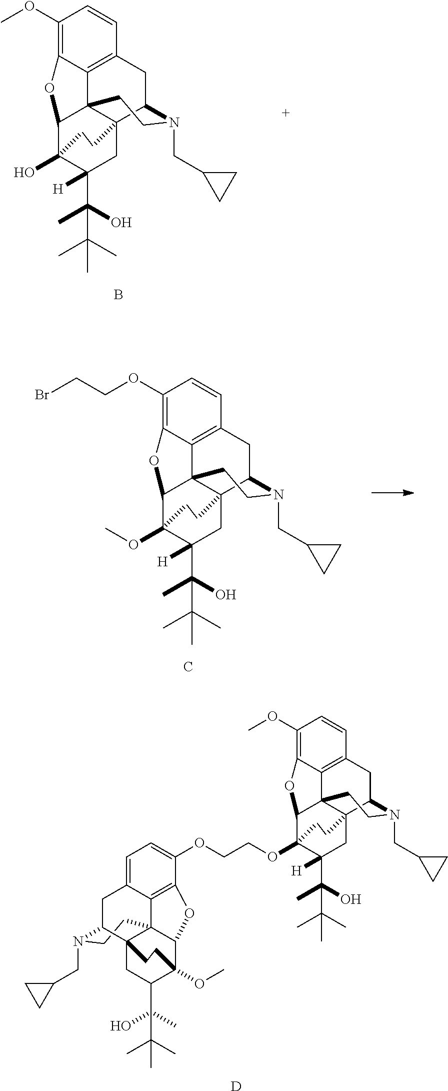

To a mixture of D-3 (58 mg, 0.12 mmol) in DMF (1.5 mL) at 0° C. was added sodium hydride (60% in mineral oil, 50.0 mg, 1.24 mmol). The mixture was stirred for 30 min at 0° C. A solution of C-2, prepared by the procedure described in the synthesis of compound C (139 mg, 0.248 mmol) in DMF (2.5 mL) was added by a syringe pump at room temperature over 5 h.

After the addition, the mixture was stirred at room temperature for 12 h. The reaction was not complete. The reaction mixture was diluted with EtOAc (100 mL). The organic phase was washed with brine, dried over magnesium sulfate, filtered, and concentrated to dryness. The crude material was purified by column chromatography (24 g, silica gel, 0 to 30% EtOAc/heptane, gradient) to afford a mixture of product and recovered D-3 (110 mg). This material was purified by reverse-phase column chromatography using MeCN/H₂O+0.05% TFA. The desired collections were concentrated to afford the title compound D (40 mg, 34%). ¹H NMR (500 MHz, CDCl₃): δ 11.80 (bs, 2H), 6.85 (d, J=8.5 Hz, 1H), 6.81 (d, J=8.0 Hz, 1H), 6.67 (d, J=8.5 Hz, 1H), 6.62 (d, J=8.5 Hz, 1H), 5.76 (bs, 1H), 5.45 (bs, 1H), 4.52 (d, J=2.0 Hz, 1H), 4.48 (d, J=2.0 Hz, 1H), 4.31-4.19 (m, 3H), 4.07-4.03 (m, 1H), 3.89-3.86 (m, 2H), 3.88 (s, 3H), 3.73-3.64 (m, 2H), 3.54 (s, 3H), 3.45-3.38 (m, 2H), 3.11-2.95 (m, 4H), 2.89-2.68 (m, 6H), 2.47-2.42 (m, 2H), 2.24-2.20 (m, 2H), 2.07-2.04 (m, 1H), 1.92-1.89 (m, 3H), 1.34 (s, 3H), 1.31 (s, 3H), 1.30-1.25 (m, 3H), 1.21-1.15 (m, 3H), 1.01 (s, 9H), 0.99 (s, 9H), 0.87-0.71 (m, 6H), 0.45-0.35 (m, 4H). MALDI MS m/z 961 [C₆₀H₈₄N₂O₈+H]⁺, Purity HPLC >99% (area %) (Detector @215 nM).

### Example 2. In Vitro Assay—MOR and KOR Binding Assay

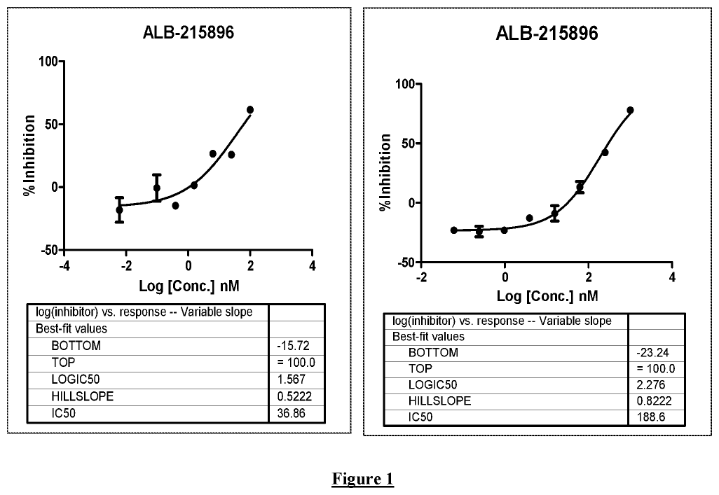

μ or κ receptor-expressing membranes were extracted by WUXi AppTec and prepared after homogenized in assay buffer (50 mM Tris, pH 7.5 with 5 mM MgCI2). 100 μl of membrane stocks were dispensed into the plates (Greiner-781946). The compounds 8 doses 4-fold serial dilution in DMSO from the Work Conc as the Compounds list with Bravo. The testing compounds were starting at 200 uM, 8-point 4-fold serial dilution in culture medium (88% DMEM, 10% FBS, 300 ug/mL G418, 2 ug/mL Blasticidin, 1% GlutaMax and 1% Pen/Strep). The reference compound 3H DAMGO (PerkinElmer) or 3H diprenorphine (PerkinElmer) were starting at 10 uM, 8-point 4-fold serial dilution. The compounds were transferred to the assay plate at 1 μl of compounds/high control/low control. 100 μl of the ratio ligand was added. The plates were sealed and shake with 300 rpm at RT for 1 hour. The Unifilter was soaked −96 GF/C filter plates with 50 μl of 0.3% PEI per well for at least 0.5 hours at RT. The reaction mixture was filtered through GF/C plates using Perkin Elmer Filtermate Harvester, and then washed each plate for 4 times with cold wash buffer. The filter was dried for 1 hour at 50° C. and the bottom of the filter plate wells were sealed using Perkin Elmer Unifilter-96 backing seal tape. They were added 50 μl of Perkin Elmer Microscint 20 cocktail. The top of filter plates was sealed with Perkin Elmer TopSeal-A sealing film. The 3H trapped on the filter was counted using Perkin Elmer MicroBeta2 Reader. The inhibition using the following equation was calculated by the following equation: % Inhibition=(1−(Assay well−Average_LC)/(Average_HC−Average_LC))*100%. The data were analyzed with Prism 5. Use the model “log (inhibitor) vs. response—Variable slope” to fit the data, as shown in FIG. 1.

### Example 3. MOR and KOR Receptor FLIPR Assay

The positive control of DAMGO and U-50488 were purchased from Sigma. The m or k Chinese Hamster Ovary cell line was constructed by WuXi AppTec and suspended in media (88% DMEM+10% FBS+300 ug/mL Geneticin+2 ug/mL Blasticidin+1% GlutaMax+1% Pen Strep).

In day 1, the culture cells were washed with 10 mL DPBS twice, added 2 mL trypsin enzyme, incubated at 37° C. for 1 minute. Then they were terminated the digestion with a complete media. The cells were centrifuged at 1000 rpm for 5 min. Gently pour off or aspirate supernatant, being careful not to aspirate cells. The cell pellet was re-suspended in 3-5 mL growth media, and 0.5 mL for cell counting was taken out. The cell viability was counted and determined by using a Vi-CELL™ (Beckman Coulter). The cell suspension to 1M cells/ml (20,000 cells per 20 μl per well) was diluted in culture medium, and seeded cells into 384 well cell plate (Greiner Cat #781280). The plates were placed in 37° C./5% CO₂ incubator for 16-20 hours.

In day 2, one vial of Fluo-4 Direct™ crystals (F10471) (Invitrogen, Cat #F10471) was thawed. The vial was added 10 ml of FLIPR Assay buffer. 0.2 ml of Probenecid was added of to each 10 ml vial of Fluo-Direct. The final assay concentration was 2.5 mM. The vial was vortexed and let stand >5 min (protect from light). The reference compounds and the testing compounds were 1:5 serially diluted in 100% DMSO for 10 pts, and then transferred 500 nL of compounds to the destination plate using Echo. Add 30 μL of assay buffer into each well of compound plate, centrifuge at 1000 rpm for 1 min. Add 20 μL per well of 2× Fluo-4 Direct™. The cell plate was loaded and buffered to a final volume of 40 μL. The cell plate was incubated for 50 min at 37° C., 5% CO₂ and 10 min at RT. The compound plate, cell plate and tip box were plated into FLIPR (Molecular Device) for reading. FLIPRTETRA protocol was running by transfer 10 μl of compounds from 384-well plate (Greiner-781280) to the cell plates. The fluorescence signal was read. The “Max-Min” starting from Read 91 to Maximum allowed were calculated. The EC50 values for each cell line using FLIPR were calculated. Using Prism to analyze the data, as shown in FIG. 2.

### Example 4. In Vitro Assay: Metabolic Stability of Compound D

The metabolic stability of Compound D in SD rat and human liver microsomes were measured One (1) μM of Compound D was incubated with SD rat or human liver microsomes with NADPH in a water bath or tecan at 37° C. for up to 60 min. All samples were monitored using LC-MS/MS.

The half-life (T_{1/2}) values of Compound D after 60 min of incubation in the presence of NADPH were 87.6 and 82.1 min, corresponding to intrinsic clearance (CLᵢₙₜ₍ₗᵢᵥₑᵣ₎) values of 28.5 and 15.2 mL/min/kg in rat and human liver microsomes, respectively. These results indicated that Compound D was moderately metabolized in SD rat and stably metabolized in human liver microsomes.

### Example 5. Stability Assay

The objective of this study was to determine the bidirectional permeability and efflux ratio of compound D in the Caco-2 cells. Caco-2 cells (purchased from ATCC) at passage number 49 were seeded on 96-well transport inserts and cultured for 22 days before being used for the transport experiment. Compound D was dosed bi-directionally at 2 μM. Samples were taken at 0 and 120 minutes after incubation and analyzed by LC-MS/MS. Compound D showed the mean apparent permeability coefficient (Pₐₚₚ) values of <0.48×10⁶ cm/s, in the apical to basolateral (A to B) direction, and <0.40×10⁻⁶ cm/s in the B to A direction. ORP-101 showed the mean apparent permeability coefficient (Pₐₚₚ) values of <0.14×10⁻⁶ cm/s, in the apical to basolateral (A to B) direction, and <0.13×10⁻⁶ cm/s in the B to A direction. Therefore, both the efflux ratios could not be calculated.

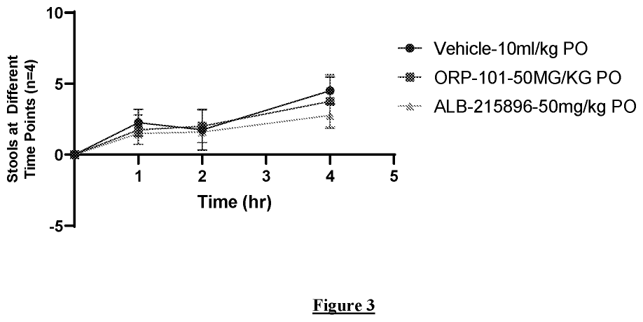

Based on the current classification criteria, compound D demonstrated low permeability across the Caco-2 cell monolayer. Considering that the Pₐₚₚ values in the B to A direction of Compound D very low (<0.5×10⁻⁶ cm/s), compound D is poor and no substrate of transporters, as shown in FIG. 3.

### Example 6: In Vivo Study—Stress Induced Fecal Output

The animals used in the studies are male CD-1 mice, average weight about 25 to 35 g, with an average of 5 mice per dose group. The mice were generally housed in colony housing where they are housed 4 per cage in polycarbonate 20 cages with access to food and water ad lib. On the day of the experiment they are transported to the procedure room where they were individually housed in metabolism cage (CAT003) after intragastric administration of Compound D at 50mpk, a reference compound of Formula II (“ORP-101”), and the vehicle alone. Compound D (“ALB-215896”) and the reference compound were each weighed and dissolved in 2.58 ml of de-ion water, mixed by vortex, to prepare the compound D and reference compound solutions at 5 mg/mL.

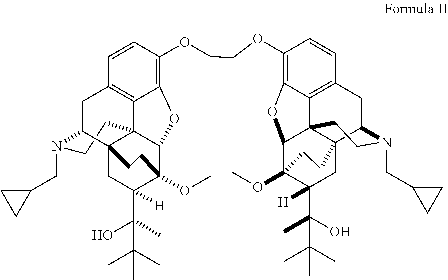

During the test, the animals were fasted with free access to water. The wire mesh bottomed tall cage creates a novel environment which induces stress in mice. Stool collection and the number of pellets excreted were determined 0, 1, 2, 4 hours after administration. Results are summarized in FIG. 3, which shows that oral doses of Compound D—50mpk significantly reduced the fecal output in mice at 1 h, 2 h and 4 h time points versus vehicle and reference compound. Details data of this study is provided below in Table 1.

TABLE 1
Stress Induce Fecal Output Result of Vehicle, ORP-101 and ALB-215896

| Group ID | Animal ID | 0 h (Stools) | 0-1 h (Stools) | 1-2 h (Stools) | 2-4 h (Stools) |
|---|---|---|---|---|---|
| Vehicle-10 ml/kg PO | Mean | 0.2 | 8.2 | 3.8 | 5.4 |
| | SEM | 0.2 | 1.46 | 0.86 | 0.68 |
| ORP-101-50 mg/kg PO | Mean | 0 | 3.8 | 3 | 2.8 |
| | SEM | 0 | 1.28 | 0.95 | 0.37 |
| ALB-215896-50 mg/kg PO | Mean | 0 | 2.4 | 1.4 | 2 |
| | SEM | 0 | 0.93 | 0.75 | 0.84 |

### Example 7: In Vivo Study—Post-Inflammatory Altered GI Transit Time

This test was designed to measure the effect of test substance on gastrointestinal hypersensitivity that occurs following inflammation. Post-inflammatory altered GI transit was induced in male CD-1 mice by injecting freshly opened oil of mustard (95% pure allyl isothiocyanate, 0.5% in ethanol). The effect of stress on GI is motility is evaluated 4 weeks later, when although there is no longer inflammation, the GI tract remains in a hypersensitive state. Effect of test substance was measured after oral administration (intragastric gavage) and subjecting the animals to environmental stress by housing them in metabolism cage. During the test, the animals were allowed access to water ad lib. The wire mesh bottomed tall cage creates a novel environment, which induces stress in mice. The number of pellets excreted was determined at 1 h, 2 h and 4 h after administration. Compound D (ALB-215896) and ORP-101 were each weighed and dissolved in 2.58 ml of de-ion water, mixed by vortex, to prepare the compound D and reference compound solutions at 5 mg/mL.

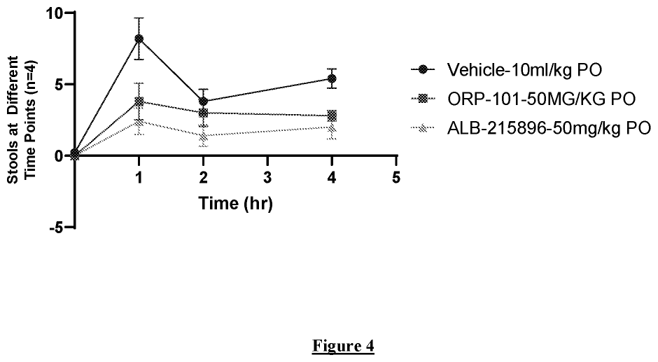

As shown in FIG. 4, Compound D-50mpk significantly decreased gastrointestinal motility at 1 h, 2 h, and 4 h time points, as measured by fecal output in post-inflammatory models. In stark contrast, the fecal output of vehicle and reference compound treated animal is significantly higher than those of Compound D treated animal, through the time points measured in the study (1 h, 2 h, and 4 h). The increased fecal output in mustard oil treated animals persisted even at 4 hours. Details data of this study is provided below in Table 2.

TABLE 2
Inflammatory Induce Fecal Output Result of Vehicle, ORP-101 and ALB-215896

| | Animal | | | | |
|---|---|---|---|---|---|
| Group ID | ID | 0 h (Stools) | 1 h (Stools) | 2 h (Stools) | 4 h (Stools) |
| Vehicle-10 ml/kg PO | Mean | 0 | 2.25 | 1.75 | 4.5 |
| | SEM | 0 | 0.95 | 1.44 | 0.96 |
| ORP-101-50 MG/KG PO | Mean | 0 | 1.75 | 2.00 | 3.75 |
| | SEM | 0 | 1.03 | 1.15 | 1.89 |
| ALB-215896-50 mg/kg PO | Mean | 0.00 | 1.49 | 1.59 | 2.77 |
| | SEM | 0.00 | 0.31 | 0.18 | 0.82 |

What is claimed is:

1. A compound of Formula (I): 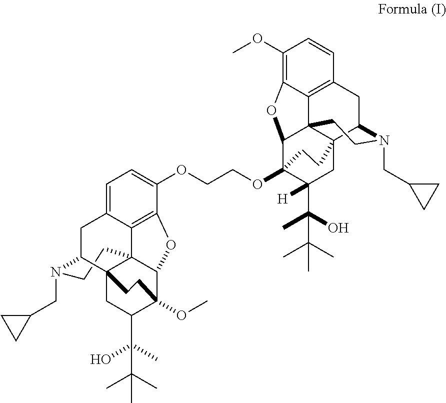
or a pharmaceutically acceptable salt, hydrate, solvate, or prodrug thereof.

2. The compound of claim 1, wherein the compound is in the form of a pharmaceutically acceptable salt.

3. The compound of claim 1, wherein the compound is in the form of a hydrochloric acid salt.

4. The compound of claim 1, wherein the compound is an opioid receptor modulator.

5. The compound of claim 4, wherein the compound is a partial agonist of a μ opioid receptor.

6. The compound of claim 4, wherein the compound is an antagonist of a κ opioid receptor.

7. The compound of claim 4, wherein the compound is a partial agonist of a μ opioid receptor and an antagonist of a κ opioid receptor.

8. A pharmaceutical composition comprising a pharmaceutically acceptable carrier or excipient and the compound of claim 1.

9. The pharmaceutical composition of claim 8, wherein said composition is formulated for oral administration.

10. The compound of claim 1, for use in the treatment, reduction of severity, or alleviation of symptom of a disease or condition associated with at least one of μ, κ, and δ opioid receptors.

11. The compound of claim 10, for use in the treatment, reduction of severity, or alleviation of symptom of a disease or condition associated with at least one of μ and κ opioid receptors.

12. The compound of claim 11, wherein the treatment, reduction of severity or alleviation of symptom of the disease or condition is by inhibiting a κ opioid receptor.

13. The compound of claim 11, wherein the treatment, reduction of severity or alleviation of symptom of the disease or condition is by activating a μ opioid receptor.

14. The compound of claim 11, wherein the treatment, reduction of severity or alleviation of symptom of the disease or condition is by inhibiting a κ opioid receptor and activating a μ opioid receptor.

15. The compound of claim 11, wherein the disease or condition is selected from the group consisting of IBS-D, visceral hyperalgesia, short bowel syndrome, or combinations thereof.

16. The compound of claim 11, wherein the disease or condition is IBS-D.

17. The compound of claim 11, wherein the disease or condition is visceral hyperalgesia.

18. The compound of claim 11, wherein the disease or condition is short bowel syndrome.

19. The compound of claim 15, characterized in that the compound is administered to a subject in need thereof at about 0.1 mg/kg of body weight to about 7.2 mg/kg of body weight.

20. The compound of claim 15, characterized in that the compound is administered to a subject in need thereof at about 10 to about 500 mg daily.
